(19) United States
(12) Patent Application Publication
(10) Pub. No.: US 2023/0250472 A1
(43) Pub. Date: Aug. 10, 2023

(54) DIGITAL METHOD FOR ANALYZING NUCLEIC ACIDS IN SAMPLES

(71) Applicant: Top Science Biotechnologies, Inc., New Taipei City (TW)

(72) Inventor: Kuo Ping CHIU, New Taipei City (TW)

(21) Appl. No.: 17/721,500

(22) Filed: Apr. 15, 2022

(30) Foreign Application Priority Data
Feb. 10, 2022 (TW) 111104836

(51) Int. Cl. C12Q 1/686; C12Q 1/6853

(52) U.S. Cl. CPC C12Q 1/686; C12Q 1/6853

(57) ABSTRACT

The present invention provides a method, entitled gene net-digital polymerase chain reaction (gn-dPCR), for the analysis of nucleic acids in a sample. It contains the following steps: (a) Perform end-repairing and 3′-A tailing to the double-stranded nucleic acid (dsNA) fragments in the sample; (b) perform a ligation reaction between the dsNA fragments with the 3′-A overhang and a double-stranded homogenous adapter with 3′-T overhang; (c) perform a pre-amplification on the dsNA fragment connected with the double-stranded homogenous adapter; (d) add an enzyme to the sample after the pre-amplification to create a nick or nicks between the double-stranded homogenous adapter and the dsNA fragment; (e) mix the sample with single type bi-direction primer (which is a constituent strand of the double-stranded homogeneous adapter), a pair of forward and reverse primers to define the boundaries of the gene net, probes associated with forward and reverse primers, together with other components required for PCR such as DNA polymerase, dNTPs, salt, etc.; (f) divide the preparation into multiple partitions. (g) perform digital polymerase chain reaction (dPCR); (h) analyze the signals in the partitions to obtain the number of positive counts of the target gene and the number of positive counts of a control gene, the ratio represents the copy number variation (CNV) of the target gene in the diseased genome; (i) additionally, by sequencing the gn-dPCR products, one can identify all the mutation sites within the defined region of the target gene; and (j) by comparing the number of reads mapped to the target gene and the number of reads mapped to the control gene, one can obtain a sequencing-derived CNV to validate the dPCR-derived CNV.

### BACKGROUND OF THE INVENTION

### 1. Technical Field

The present invention relates to a method for analyzing nucleic acids in samples and a kit thereof, which is useful to solve the problem that the cell-free nucleic acid (cfNA) samples are fragmented easily and may not include two PCR primer binding sites required for existing digital polymerase chain reaction (dPCR). The method can be used to amplify and sequence the specific gene fragments of different sizes in a nucleic acid sample to improve the sensitivity for analysis. Using the analyzed sequencing results, one can overcome the limitation of the existing dPCR technology, which is not applicable to analysis of cell-free DNA (cfDNA) in body fluids, and can break the limit of the maximum detectable number of mutations (≤4).

### 2. Description of Related Art

Digital polymerase chain reaction (dPCR) is a highly sensitive bioanalytical technique, which has been increasingly used in genomics analysis to analyze gene copy number variations (CNVs) and gene mutations.

Currently, both droplet-based (ddPCR), titer plate-based, and chip-based dPCR methods have been commercialized, among which the droplet-based dPCR method is more tolerant and easy to operate than other similar analysis methods, especially the QX200 droplet digital PCR (ddPCR) system sold by Bio-Rad. QX200 is an advanced ddPCR analytical instrument which combines microfluidics and surfactant chemistry to separate PCR into water-in-oil droplets, enables absolute nucleic acid (NA) quantification of samples, and allows droplets of nearly 20,000 nanoliter size per sample to be performed at a time, so that makes it becomes one of the most efficient analytical instruments of its kind. Therefore, the method provided in this invention uses the QX200 system for testing, but the principle is applicable to various dPCR methods.

Similar to other dPCR methods, the QX200 ddPCR instrument uses a pair of PCR primers that can specifically bind to the target gene to perform PCR amplification in the sample, together with a probe that can specifically bind to the region between the primers for detecting target nucleic acids. The instrument dilutes and partitions the samples in order to separate nucleic acid fragments for separate PCR reactions which contain single or very limited number of target nucleic acid molecules. In this way, the ddPCR can effectively determine whether the fluorescent signal reaction in each partition is negative or positive, and then based on the poisson distribution and the number of positive and negative reactions to estimate the number of nucleic acid sequences in the original sample. Among them, the total number of positive droplets is closely related to the total number of target nucleic acid molecules in the samples, so by comparing the biological properties of different samples (e.g. the number of copies or mutation sites), the biomedical significance of each biological property can be determined.

### BRIEF SUMMARY OF THE INVENTION

Although existing dPCR techniques are suitable for the analysis of gDNA samples, they are not suitable for cfNA samples, including samples of cfDNA and cell-free RNA (cfRNA). Because cfNA will break into fragments in a random or at least near-random, it may not be possible to contain specific sites within the same fragment that allow both primers to bind simultaneously. So that the existing dPCR has poor sensitivity in the analysis of cfNA samples and is prone to false negative results. In addition, the number of cfNA is small and is easy to be lost during the experiment, therefore greatly increase the difficulty of cfNA sample analysis.

Moreover, cfNA samples are non-invasive genetic materials which are easy to obtain and are ubiquitous in body fluids, so they have become more and more popular in medical examinations for diagnosing various diseases. Thus, how to effectively overcome the problems of low quantity and random fragmentation of cfNA samples, so to improve the sensitivity and accuracy of analysis and reduce false negatives have become a major challenge in this field.

In view of this, in order to solve the above-mentioned problems of the existing dPCR technology, the present invention provides a method for analyzing nucleic acids in samples entitled gene net-digital polymerase chain reaction (gn-dPCR) and verified with Bio-Rad's QX200 ddPCR operating system. Through the usage of double-stranded homogeneous adapter and pre-amplification, the present invention firstly solves the problems that the total amount of the cfNA sample may not be sufficient for analysis and that the fragmented cfNA sample may not contain both primer binding sites. Then through usage of the upstream forward primer and downstream reverse primer to a specific gene to be investigated, boundary of the specific gene can be defined as a “gene net”, making almost all the fragments within the net to be replicated and detected. All of the mutation sites' information can be further analyzed by sequencing, and the CNV value (i.e., copy number of an oncogene vs. copy number of a control gene) directly obtained by the ddPCR instrument can be verified by utilizing the relative proportion of sequence reads respectively mapped to the oncogene and the control gene (theoretically should be the same). In this way, it can not only improve the analytical sensitivity and reduce the false negative results of cfNA samples, but can also sequence specific gene fragments of different sizes in specific regions of nucleic acid samples to obtain all mutation points and verify the CNV values, while the aforementioned advantages of the subject application cannot be achieved by traditional methods.

In particular, one main aspect of the present invention is to provide a method for analyzing nucleic acids in samples, wherein the samples contain one or more double-stranded nucleic acid (dsNA) fragments. The method comprises the steps of: (a) forming a dsNA fragment with 3′-A overhang by adding 3′-A tail to the dsNA fragment(s) in the samples; (b) performing a ligation reaction between the dsNA fragment with the 3′-A overhang and a double-stranded homogenous adapter to form a dsNA fragment connected with the double-stranded homogenous adapter, wherein the double-stranded homogenous adapter is a complementary dsNA fragment having one oligonucleotide strand with 5′-phosphate and the other oligonucleotide strand with 3′-thymine (T) or 3′-uracil (U); (c) performing pre-amplification on the dsNA fragment connected with the double-stranded homogenous adapters; (d) adding an enzyme to the samples after the pre-amplification to create a nick at 3′-end of the double-stranded homogenous adapters on the dsNA fragment; (e) after mixing the samples as well as required components for dPCR with a monotype bidirectional primer constituting an oligonucleotide of the double-stranded homogeneous adapter, diluting, dividing the same into multiple partitions, and performing dPCR, such that heating during dPCR causes the strand with the nicks to fall off, and (f) receiving signal results provided by a probe in each partition.

In one or more embodiments, the step (e) further comprises adding a forward primer and a reverse primer both specific to a target gene, and probes corresponding to the forward primer and the reverse primer.

In one or more embodiments, the probing system contains multiple probes targeting different locations of the gene to be investigated.

In one or more embodiments, the forward primer and the reverse primer are designed to specifically bind to the boundaries of a defined range of the target gene.

In one or more embodiments, the samples are obtained from body fluid of an organism.

In one or more embodiments, the dsNA fragment in the samples is cfDNA or cfRNA-derived cDNA.

In one or more embodiments, an end of the double-stranded homogenous adapter in the step (b) is 3′-T overhang or 3′-U overhang. Usage of 3′-T overhang or 3′-U overhang can be selected as needed.

In one or more embodiments, the double-stranded homogenous adapter cannot self-ligate.

In one or more embodiments, the enzyme in the step (d) is an uracil-specific excision reagent enzyme (USER enzyme).

In one or more embodiments, the PCR in step (e) is performed by droplet-based, titer plate-based PCR, or other dPCR.

In one or more embodiments, the method further comprises the step of (g): identifying mutations in all fragments by sequencing. In one or more embodiments, the method further comprises the step of (h): after sequencing, the relative sequence read numbers (for example, the read number of an oncogene and the read number of a normal gene both of which originated from the genome of a cancer) are compared to obtain a value of copy number variation (CNV) for the oncogene in the cancer. Besides, in one or more embodiments, the method further comprises the step of (i): verifying the CNV of positive counts of oncogenes to positive counts of normal genes obtained in step (f) with a CNV result obtained in step (h).

In one or more embodiments, the dsNA fragments in the samples are derived from single-stranded nucleic acid (ssNA). Besides, in other embodiments, the ssNA in the samples is RNA-derived cDNA. In a preferable embodiment, steps (a) and (b) of the present invention method are omitted when the ssNA is RNA-derived cDNA, and satisfied with the following condition that when forming the dsNA fragment from the ssNA which is derived from RNA, and both ends of the dsNA fragment are ligated with a double-stranded homogenous adapter, which is a complementary dsNA fragment having one oligonucleotide strand with 5′-phosphate and the other oligonucleotide strand with 3′-thymine (T) or 3′-uracil (U).

A main aspect of the present invention is to provide a kit for performing the method as mentioned above. The kid comprises: (i) a double-stranded homogenous adapter as defined in the present invention; (ii) primers, comprising a monotype bidirectional primer corresponding to the double-stranded homogeneous adapter, and a forward primer and a reverse primer both specific to a target gene; (iii) probes, comprising probes corresponding to the forward primer and the reverse primer, and a plurality of probes comprising different mutation sites; (iv) enzymes, comprising uracil-specific excision reagent enzyme (USER enzyme); (v) PCR reagents; and (vi) detection reagents.

Compared with the conventional nucleic acid analysis technology, the advantages of the present invention are:

1. By performing pre-amplification, the present invention can ensure that the amount of nucleic acid samples is sufficient for use in various purposes, such as sequencing, stock preparation, data validation, and the like.

2. The sensitivity of analyzing nucleic acid samples according to the invention is twice higher than that of the conventional dPCR technology (e.g. ddPCR of Bio-Rad used herein).

3. The present invention can aim to amplify the specific gene fragments of different sizes in nucleic acid samples.

4. In the invention, by utilizing the monotype bidirectional primer corresponding to the double-stranded homogeneous adapter, the upstream forward primer and the downstream reverse primer, various fragments of different sizes between the given regions in connection with the specific genes in nucleic acid samples can be sequenced to find out all mutations. After sequencing, all mutation sites can be obtained by comparing the sequence of diseased genes with the sequence of normal genes. Furthermore, the relative cfDNA proportion of two genes in body fluid can be obtained by mapping followed by comparing the read numbers of two different gene sequences.

5. The present invention increases the detectable amounts of gene mutations limited by the existing dPCR technology, and thus is beneficial to discover new gene mutants.

### BRIEF DESCRIPTION OF THE SEVERAL VIEWS OF THE DRAWINGS

In order to better understand the technical content of the present invention, the accompanying drawings show preferred embodiments of the present invention. However, it should be understood that the present invention is not limited to the technical contents shown in the accompanying drawings.

FIG. 1 shows a schematic flowchart of the present invention.

FIG. 2 shows a schematic diagram of the comparison between the present invention method and the Bio-Rad ddPCR analysis method.

FIG. 3 shows the comparative analysis results of the positive counts of the ctDNA fragments of the N-myc gene of neuroblastoma (NB) patients analyzed by gn-ddPCR of the present invention.

FIG. 4 (figure changed) shows a schematic diagram of the difference between the method of the present invention and the conventional dPCR method.

### DETAILED DESCRIPTION OF THE INVENTION

The following provides a detailed description on the embodiments of the present invention. However, such description and the embodiments provided shall not be used to limit the scope of the present invention. Any modification and change made by a person with ordinary skills in the technical field of the present invention based on the embodiments disclosed by the present invention and within the principle and scope of the present invention shall be treated to be within the scope of the present invention.

The term “one” or “a” described in the following content shall mean one or more than one, i.e., at least one.

The term “comprising, having or including” described in the following content shall mean the existence of one or more than one parts, steps, operations and/or elements or the inclusion of such parts, steps, operations and/or elements.

The term “approximately or about” or “basically” described in the following content shall mean that a certain value or range is close to an acceptable specified tolerance, and the purpose of the use of such term is to prevent a third party's unreasonable, illegal or unfair interpretation of a value or range disclosed by the present invention to be within or equivalent to the exact or absolute value or range disclosed by the present invention only.

One main purpose of the present invention is to provide a method for analyzing nucleic acids in samples, wherein the samples contain one or more double-stranded nucleic acid (dsNA) fragments. The method comprises the steps of: (a) forming a dsNA fragment with 3′-A overhang by adding 3′-A tail to the dsNA fragment(s) in the samples; (b) performing a ligation reaction between the dsNA fragment with the 3′-A overhang and a double-stranded homogenous adapter to form a dsNA fragment connected with the double-stranded homogenous adapter, wherein the double-stranded homogenous adapter is a dsNA fragment made of one oligonucleotide strand with 5′-phosphate and the other oligonucleotide strand with 3′-thymine (T) or 3′-uracil (U); (c) performing pre-amplification for the dsNA fragment connected with the double-stranded homogenous adapters; (d) adding an enzyme to the samples after the pre-amplification to create nicks at U bases on the double-stranded homogenous adapters on the dsNA fragment; (e) after mixing the samples as well as required components (such as but not limited to dNTPs, upstream forward primer, upstream probe, downstream reverse primer, downstream probe, enzymes, and the like) for dPCR with a monotype bidirectional primer constituting an oligonucleotide of the double-stranded homogeneous adapter, and performing dPCR, such that heating from the dPCR causes the strand with nicks to fall off, and (f) receiving signal results provided by a probe in each partition. Wherein, the dPCR can be ddPCR, which is performed by mixing the sample in step (e) with all the components required for PCR reaction, diluting, mixing with oil to form micro-droplets, and ddPCR was performed within each droplet.

Another main purpose of the present invention is to provide a kit for performing the method of the present invention, comprising: a double-stranded homogenous adapter as defined in the present invention; (ii) primers, comprising a monotype bidirectional primer corresponding to the double-stranded homogeneous adapter, and a forward primer and a reverse primer both specific to a target gene; probes, comprising probes corresponding to the forward primer and the reverse primer, and a plurality of probes comprising different mutation sites; enzymes, comprising uracil-specific excision reagent enzyme (USER enzyme); PCR reagents; and detection reagents.

The term “samples” as used herein refers to body fluid samples, tissue samples, forensic samples, or fossil samples of an organism comprising one or more dsNA fragments, preferably from body fluid sample of an organism. The dsNA fragment is preferably cfNA, such as cfDNA or cfRNA. The dsNA fragment contains one or more genetic mutations or single nucleotide polymorphism (SNP), such as but not limited to: point mutations with a single base change, including synonymous mutation, silent mutation, missense mutation, frameshift mutation, nonsense mutation; or large mutation of multiple base changes, including deletion, rearrangement, and insertion, wherein rearrangement mutations include duplication, inversion, and translocation. The organism includes mammal and non-mammal, such as but not limited to: human, non-human primate, sheep, dog, murine rodent (e.g. mouse, rat), guinea pig, cat, rabbit, cow, horse; the aforementioned non-mammal such as but not limited to: chicken, amphibian and reptile. The organism is preferably human in the present invention. The body fluid sample is for example but not limited to blood, saliva, urine, tears, cerebrospinal fluid, various secreted mucus and so on.

The term “double-stranded homogenous adapter” as used herein refers to a dsNA fragment formed by two complementary strands. One strand of the complementary dsNA fragment is an oligonucleotide with 5′-phosphate, and the other strand is an oligonucleotide with 3′-thymine (T) or 3′-uracil (U). The 3′-thymine (T) or 3′-uracil (U) is an overhang. The complementary dsNA fragment cannot self-ligate. The term “homogenous” means that the adapters are of a single kind. The term “adapter” refers to an oligonucleotide for which the double-stranded homogenous adapter can be ligated to the ends of dsNA molecules in a sample. A double-stranded homogenous adapter may be 10 to 50 bases in length, preferably 10 to 30 bases in length, and more preferably 10 to 20 bases in length. If the length is shorter than 10 nucleotides, the specificity for annealing may be reduced. Additionally, it may not be cost-effective when the length is longer than 20 nucleotides.

If the sample used in the method of the present invention contains RNA, the RNA can be converted into cDNA. That is, the dsNA fragment in the present invention is derived from ssNA, and the ssNA is RNA-derived cDNA. In a preferred embodiment, after converting the RNA into ssNA and then the final dsNA fragment, both ends of the obtained dsNA fragment have the double-stranded homogenous adapter as defined in the above content. The method of converting the RNA into the cDNA and forming the dsNA fragment with the double-stranded homogenous adapter at both ends can refer to the contents of WO2020/237010A1, the present invention incorporated by reference herein. If this method is adopted, since the obtained dsNA fragment already has the double-stranded homogenous adaptors at both ends, it is unnecessary to perform steps (a) and (b) of the method of the present invention, so that step (c) can be directly performed.

The term “pre-amplification” as used herein means amplifying the amounts of dsNA fragments in samples beforehand and making all dsNA fragments in samples have double-stranded homogeneous adapters before PCR reaction.

The term “enzyme” as used herein refers to a protein that can carry out a biochemical reaction to create a nick, or nicks, on a double-stranded homogenous adapter ligated to a dsNA fragment. After the dsNA fragment with a nick is processed by PCR heating, one strand with the nick, or nicks, falls off to form a dsNA fragment with 3′-overhang. The enzyme in the invention may be, but not limited to, uracil-specific excision reagent enzyme (USER enzyme), which can create nicks at the uracil position. USER enzyme is a mixture of uracil DNA glycosylase (UDG) and DNA glycosylase-lyase Endo VIII. UDG catalyzes the cleavage of uracil bases to form a de-base (de-uracil) site, but keep the phosphodiester skeleton intact. The lyase activity of Endo VIII causes the phosphodiester bonds at the 3′- and 5′-ends of the de-base site to break and release base-free deoxyribose.

The term “monotype bidirectional primer” as used herein means that one oligonucleotide constituting a double-stranded homogenous adapter can be used alone as a forward and reverse primer (so-called “bidirectional” herein) provided that the dsNA in a sample has a double-stranded homogeneous adapter. It serves as the primer to anneal forward and reverse strands and guide polymerase to perform polymerization in the elongation step.

The term “desired probe” as used herein refers to a probe complementary to one strand of a dsNA fragment. The probe may be, for example, but not limited to, a radioactive probe (e.g. an isotope 32P probe, an isotope 3H probe, an isotope 35S probe, etc.), a non-radioactive probe (e.g. a biotin probe, a digoxigenin probe, etc.), and a fluorescent probe. In the present invention, the probe is preferably a fluorescent probe.

In one or more embodiments of the present invention, the step (a) further comprises end-repairing and 3′-A tailing reaction to the dsNA fragment in samples. The steps of end-repairing and 3′-A tailing can be performed by conventional methods or kits, such as NEBNext Ultra End Repair/dA-Tailing Module (NEB, E7442S/L).

In one or more embodiments of the present invention, the step (e) further comprises adding a forward primer and a reverse primer both specific to a target gene, and also probes corresponding to the forward primer and the reverse primer.

The aforementioned “forward primer” and “reverse primer” refer to a primer specific to the target gene of a dsNA fragment in samples. A forward primer is a primer that can specifically bind to the reverse strand of a dsNA fragment, whereas a reverse primer is a primer that can specifically bind to the forward strand of a dsNA fragment. Moreover, the forward primer and the reverse primer are designed corresponding to the ends of a defined region on the target gene. The defined range may be adjusted and established according to the purpose of analysis. The term “probes corresponding to the forward primer and the reverse primer” refers to a probe that can release substances emitting signals under detection (including, but not limited to, fluorescence).

In one or more embodiments of the present invention, the PCR in the step (e) is performed by droplet-based, titer plate-based, or chip-based dPCR.

In one or more embodiments of the present invention, step (f) is performed by photography and image analysis and accessing techniques, such as, but not limited to, using a capillary accompanying a laser detector.

In one or more embodiments of the present invention, the method further includes step (g), which comprises identifying mutations in all fragments by sequencing, and validating a CNV value obtained from dPCR through the relative sequence read numbers between two genes. Wherein the sequencing method is for example, but not limited to, Sanger sequencing, NGS next-generation sequencing (e.g. Illumina), single molecule sequencing (e.g. Nanopore and PacBio), Ion Torrent sequencing and so on.

The following description of the present invention is the necessary technical contents which can be easily understood by those with ordinary skill in the art. If the present invention is varied and modified to suit different uses and conditions without violating the spirit and scope thereof, other embodiments may still fall into the scope of the present invention.

### Examples

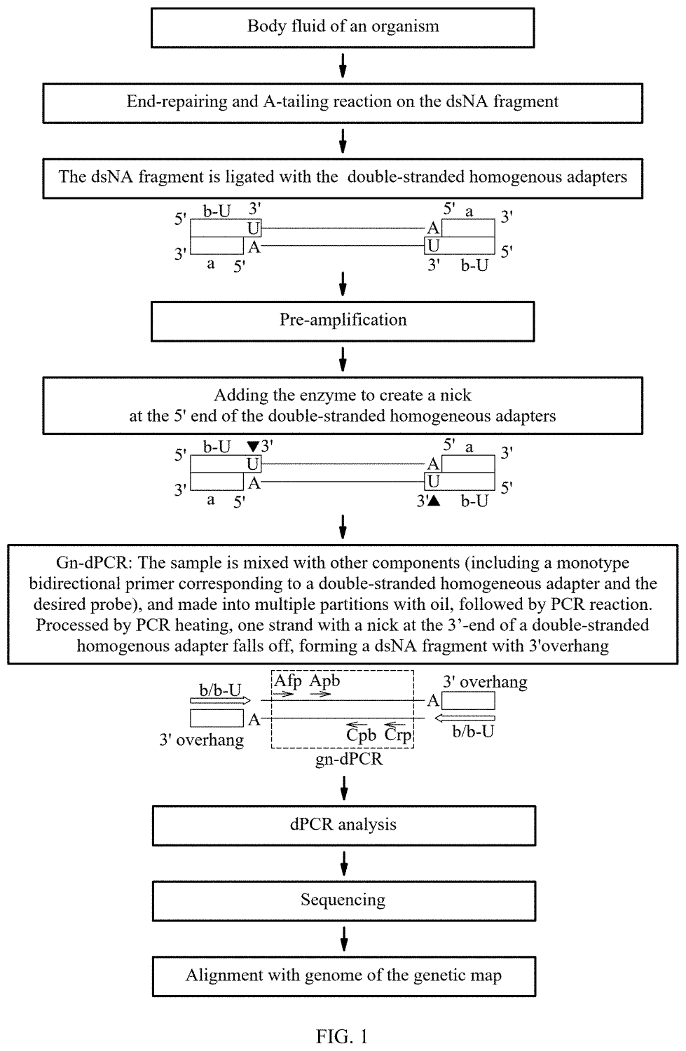

Reference is made to FIG. 1, which illustrates a flowchart of the method of present invention. In FIG. 1, “Afp” represents a forward primer (SEQ ID NO: 1); “Apb” represents a probe corresponding to the forward primer (forward probe, SEQ ID NO: 2); “Crp” represents a reverse primer (SEQ ID NO: 3); “Cpb” represents a probe corresponding to the reverse primer (reverse probe, SEQ ID NO: 4). A double-stranded homogeneous adapter is formed by annealing “a (SEQ ID NO: 5)” with “b (SEQ ID NO: 6)” or “a” with “b-U (SEQ ID NO: 7)” (“a”, “b” and “b-U” are oligonucleotides). “b” or “b-U” indicated by the arrows in the figure (shown as b/b-U) indicates a corresponding monotype bidirectional primer to the double-stranded homogeneous adapter. “b-U” has the same sequence as “b”, except that “T” (can be more than one) in the sequence is replaced by “U”. Either b or b-U can work as a monotype bidirectional primer, but only b-U should be used if sequencing will be performed later. The primers used for pre-amplification are “a” and “b-U”, because only “U” can be cleaved by USER enzyme.

[End-Repairing and A-Tailing Reaction]

The sample (e.g. dsNA fragments in body fluid of an organism) is subjected to end-repairing and 3′-A tailing. The dsNA fragments thus have 3′-A overhang. The end-repairing and 3′-A tailing reactions can be performed by conventional methods or kits, such as NEBNext® Ultra End Repair/dA-Tailing Module (NEB, E7442S/L).

[Ligation of Double-Stranded Homogeneous Adapters]

The end-repaired and 3′-A tailed dsNA fragment is ligated with a double-stranded homogeneous adapter via ligase in a ligation buffer and appropriate ligation mixture to form a dsNA fragment connected with a double-stranded homogeneous adapter, in which a 3′-thymine (T) or 3′-uracil (U) overhang of the double-stranded homogeneous adapter is connected to a 3-A overhang of the dsNA fragment. The ligated mixture can be used for PCR amplification. Optionally, the dsNA fragment may be purified prior to subsequent processing.

The double-stranded homogeneous adapter is formed by annealing two single-stranded complementary nucleic acid fragments. One of the single-stranded nucleic acid fragment has a 5′-phosphate group, and the other has a 3′-thymine (T) or 3′-uracil (U). The 3′-thymine (T) or 3′-uracil (U) is an overhang. The scheme of the double-stranded homogeneous adapter is designed with reference to Taiwan Patent Application Publication No. TW202035699A, which is incorporated herein by reference (make sure to incorporate this as ref).

[Pre-Amplification]

Before performing PCR on the dsNA fragments ligated with double-stranded homogeneous adapters, amplify the sample in advance to increase the amount of dsNA fragments. In such pre-amplification, the 3′-U-containing oligonucleotide is used as primer.

[Formulation of Nick]

After pre-amplification, an enzyme is added to the sample, and thereby one or more nicks are formed on the dsNA fragment, especially the one at the 3′-end of a double-stranded homogenous adapter.

The enzyme is used to create a nick on each U site. After the dsNA fragment with nick(s) is heated during PCR, the strand with the nick at the 3′-end of the double-stranded construct falls off to form a dsNA fragment with 3′-overhang.

[Gene Net-dPCR]

After the nucleic acid molecules in the sample are mixed with various components required for dPCR reaction including dNTPs, DNA polymerase, monotype bidirectional primers, gene-specific forward and reverse primers, probes corresponding to the forward and reverse primers. The sample mixture is then partitioned and gn-dPCR is performed. The reaction result can be analyzed by QX200 instrument.

During heating in dPCR reaction, the strand with a nick at the 3′-end of the double-stranded homogenous adapter falls off, such that a dsNA fragment originally with a double-stranded homogeneous adapter becomes a fragment with 3′-overhang, while heating also causes the dsNA fragments to become single-stranded nucleic acid (ssNA) fragments. It is beneficial for a monotype bidirectional primer to complementarily anneals to the 3′-overhang regions of forward and reverse strands of the dsNA fragment after thermal separation, resulting in a primer extension reaction.

During annealing and extension step of gn-dPCR reaction, the desired probes and the forward and reverse primers specific to the target gene also specifically bind to their specific binding sites in the heat-denatured nucleic acid fragments. The forward primers and the reverse primers are designed to be specifically bound to the ends of a defined range as desired for a target gene. The upstream forward primer and the downstream reverse primer define a “net” for the target gene with clear boundaries. The distance between a forward primer and a reverse primer can be set and adjusted according to the purpose of analysis. The forward primers and the reverse primers “prime” extension reactions to form dsNA fragments from heat-denatured ssNA fragments, of which may contain various mutation sites within the “gene net”. The mutations within the gene net can be detected by sequencing. The prerequisite for sequencing is that the dPCR products must be large enough in size and sufficient in quantity for sequencing library preparation, while the traditional dPCR products are normally small in size and may not sufficient in quantity for the purpose of sequencing. The gn-dPCR products, which contain almost all of the fragments within the net, are large in both size and quantity and are thus suitable for sequencing. In theory, either aliquots or samples recovered from dPCR can be used for sequencing.

[Signal Strength Results Receiving]

Partitions after dPCR reaction are evaluated by instrument (e.g. QX200) to determine whether the fluorescent signal from each partition is positive or negative.

[DNA Sequencing]

Before dPCR reaction, the sample is divided into two aliquots. One aliquot is subjected to dPCR followed by signal analysis, and the other aliquot is subjected to DNA sequencing. Sequencing is able to reveal all mutations in every fragment within the gene net, and mutations in all fragments can be combined to represent all mutations within the defined gene net region. The sequencing method can be but not limited to Sanger sequencing, NGS (next-generation sequencing, e.g. Illumina), single molecule sequencing (e.g. Nanopore or PacBio), Ion Torrent sequencing and so on. By sequencing the gn-dPCR amplified products, all mutation sites in the target gene (e.g., an oncogene) can be identified. Furthermore, a sequencing-based CNV can also be obtained by comparing the sequence read numbers respectively mapped to the gene of interest (i.e., the experimental gene such as an oncogene whose copy number may increase along with cancer development) and a stable gene (i.e., the control gene whose copy number is known to remain unaltered by cancer development). The sequencing-based CNV value obtained above can thus be compared to, and thus to evaluate, the dPCR-based CNV value obtained by counting the positive fluorescent signals or by other labeling methods.

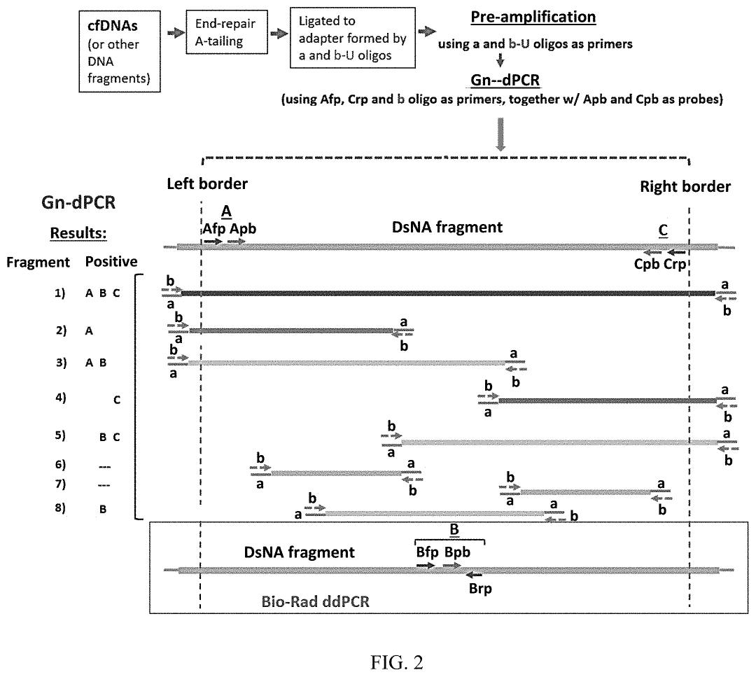

FIG. 2 is a schematic diagram showing the comparison between the present invention and the Bio-Rad ddPCR method in prior arts. The grey bar shown on top represents the region of investigation in the target gene (i.e., N-myc oncogene in this case). The A and C domains that define the boundaries of gene net are also labeled. In parallel, the grey bar shown on bottom represents the same region in N-myc oncogene, and the region targeted by Bio-Rad ddPCR detection system is labeled in the middle of the bar (domain B). Potential fragments are shown between. After random fragmentation, a cfNA fragment in the sample may turn into Fragments 1-8. The Bio-Rad ddPCR analysis method will detect Fragments 1, 3, 5, and 8, as these fragments contain the region covered by the forward primer (Bfp, SEQ ID NO: 8), the reverse primer (Brp, SEQ ID NO: 9) as well as the corresponding probe (Bpb, SEQ ID NO: 10), all of which essential for generating fluorescence signals. On the other hand, Fragments 2, 4, 6, and 7 cannot be amplified and thus cannot be detected, causing false negatives to occur. In contrast, for the method of the present invention, all the fragments contain double-stranded homogenous adapters of “a” sequence, allowing the monotype bidirectional primer of “b” or “b-U” sequence to anneal. The fragments having “Afp” and/or “Crp” binding sites (i.e. Fragments 1-8, except Fragments 6 and 7) can be annealed and probed by “Apb” and/or “Cpb”, respectively. Hence, most of the fragments can be amplified by gn-dPCR (gn-ddPCR in this case), so to generate fluorescent signals under detection. (Notice that, Fragments 6 and 7 can also be detected if probes are provided for the corresponding regions). This approach is coined as “gene net” in the present invention, specifically designed to capture almost all the possibilities effectively. Thus, the gene net approach should be able to improve the sensitivity and the accuracy of dPCR analysis. Furthermore, the gene net defined by “Afp”, “Apb”, “Crp”, “Cpb”, and b/b-U allows us to detect various mutation sites within the net when an aliquot of the same sample is subjected to sequencing. Sequencing requires sufficient amount of sample, which is already satisfied by the pre-amplification step prior to dPCR. As such, through sequencing and mapping the sequence reads to human genome assembly, all mutations within the gene net can be identified (for a patient, if clinical sample are analyzed), and the dPCR-derived CNV can be validated by sequencing-based CNV value generated through comparing the ratios of reads mapping respectively to the experimental and control genes.

[Experimental Results]

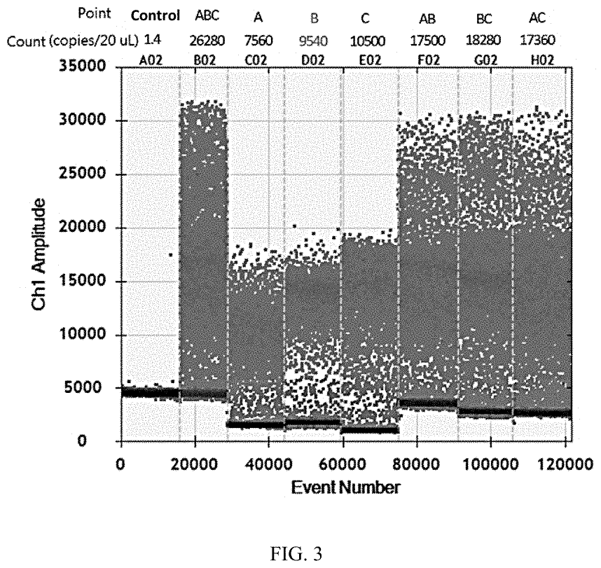

Continuing to FIG. 3, where shows ddPCR results for all possible combinations of A, B and C. During the ddPCR, all probes is labeled with FAM, and analysis is performed by QX200 machine. Independently, “A” utilizes Afp+b+Apb, “B” utilizes the Bio-Rad method (Bfp+Brp+Bpb), and “C” utilizes Crp+b+Cpb (see FIG. 2). For a combination of any two among A, B and C, “AB” utilizes Afp+b+Apb+Bfp+Brp+Bpb, “AC” utilizes Afp+b+Apb+Crp+Cpb (this combination is the gn-dPCR method of the present invention), and “BC” utilizes Bfp+Brp+Bpb+Crp+b+Cpb. For a combination of all three, “ABC” utilizes Afp+b+Apb+Bfp+Brp+Bpb+Crp+Cpb. Results show, as expected: 1) numbers of positive counts of one-selected combinations are quite similar; 2) numbers of positive counts of two-selected combinations are also very similar; 3) furthermore, the number of positive counts for any two-selected combination is about twice the number of positive counts of any one-selected combinations; and 4) furthermore, number of positive counts of three-selected combination is about three times that of any one-selected combination. Thus, the results clearly indicate that the gn-dPCR method of the present invention (AC) is able to double the sensitivity, comparing to the Bio-Rad method (B).

It can be seen from above that, comparing to the traditional ddPCR analysis method, the gn-dPCR method of the present invention increases the analytic sensitivity for cfNA samples and reduces the occurrence of false negatives significantly.

Additionally, it can be seen that the gn-dPCR method of the present invention successfully overcomes the potential problem that may result from insufficient quantity of a sample for accurate analysis.

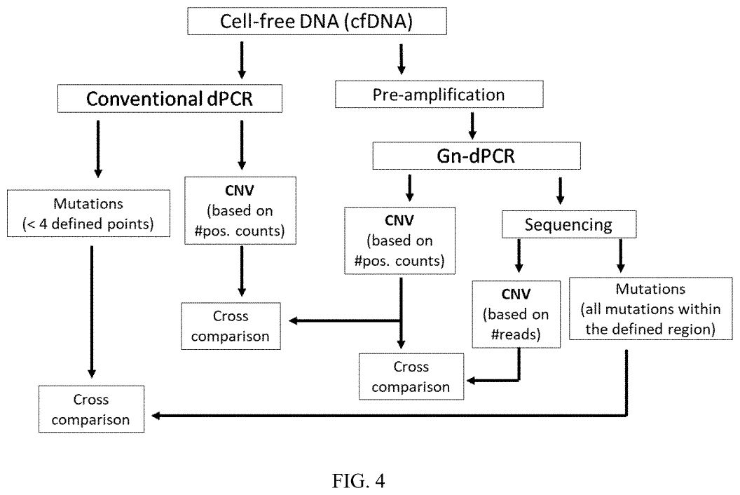

In summary, the present invention is able to successfully resolve a number of problems frequently associated with cfNA analysis. These include the following issues: 1) potentially insufficient sample quantity for dPCR and/or sequencing analysis; 2) false negatives that tightly associated with conventional dPCR methods; 3) absence of a method for independent further validation of dPCR-derived CNV value; 4) limited number of mutation sites that can be detected by traditional dPCR method. As shown in FIG. 4, comparing to the conventional dPCR technology, as demonstrated by ddPCR in this case, the present invention is able to amplify and sequence gene-specific nucleic acid fragments of different sizes in body fluids to improve sensitivity (e.g., by including fragments with single or with no primer binding sites into analysis), accuracy (e.g., by removing most false negatives), completeness (e.g., by recruiting all mutation sites within the gene net for further analysis) and comprehensiveness (e.g., providing an independent method for dPCR-based CNV estimation). Thus, the present invention not only provides support to enhance the existing functions of traditional dPCR methods, but also suggests new opportunities for dPCR technologies.

The above is the detailed description of the present invention. However, the above is merely the preferred embodiment of the present invention and cannot be the limitation to the implement scope of the invention, which means the variation and modification according to the present invention may still fall into the scope of the present invention.

1. A method for analyzing nucleic acids in samples, wherein the samples contain one or more double-stranded nucleic acid (dsNA) fragments, the method comprising the steps of:
(a) forming a dsNA fragment with 3′-A overhang by adding a 3′-A tail to the dsNA fragment(s) in the sample;
(b) performing a ligation reaction between the dsNA fragment with the 3′-A overhang and a double-stranded homogenous adapter to form a dsNA fragment connected with the double-stranded homogenous adapter, wherein the double-stranded homogenous adapter is a complementary dsNA fragment having one oligonucleotide strand with 5′-phosphate and the other oligonucleotide strand with 3′-thymine (T) or 3′-uracil (U);
(c) performing pre-amplification on the dsNA fragment connected with the double-stranded homogenous adapters;
(d) adding an enzyme to the samples after the pre-amplification to create a nick or nicks at or near the 3′-end of the double-stranded homogenous adapters on the dsNA fragment;
(e) after mixing the samples with required components for digital polymerase chain reaction (dPCR) and a monotype bidirectional primer that constitutes an oligonucleotide for the double-stranded homogeneous adapter, followed by dilution and division of the sample into multiple partitions, dPCR is conducted, such that heating during dPCR causes one strand with the nick at, or near, the 3′-end of the double-stranded homogeneous adapter to fall off, and
(f) receiving signal results provided by probes from each partition.

2. The method of claim 1, wherein the step (e) further comprises adding a forward primer and a reverse primer both specific to a target gene, and probes corresponding to the forward primer and the reverse primer.

3. The method of claim 2, wherein the probes are a plurality of probes comprising different mutation sites.

4. The method of claim 2, wherein the forward primer and the reverse primer are designed to specifically bind to the ends of a defined range in the target gene.

5. The method of claim 1, wherein the samples are obtained from any body fluid of an organism.

6. The method of claim 1, wherein the dsNA fragment in the samples is cell-free DNA (cfDNA), cell-free RNA (cfRNA), forensic DNA, or fossil DNA.

7. The method of claim 1, wherein an end of the double-stranded homogenous adapter in the step (b) is 3′-T overhang or 3′-U overhang.

8. The method of claim 7, wherein the double-stranded homogenous adapter does not self-ligate.

9. The method of claim 1, wherein the enzyme in the step (d) is an uracil-specific excision reagent enzyme (USER enzyme).

10. The method of claim 1, wherein the PCR in the step (e) is performed by droplet-based, titer plate-based, or chip-based digital PCR.

11. The method of claim 1, further comprising the step of (g): identifying mutations in all fragments by sequencing.

12. The method of claim 11, further comprising the step of (h): after sequencing, the relative copy number variation (CNV) for target genes and normal genes in the genome of source cancer cells is compared with relative sequence read numbers of the target genes and the control genes.

13. The method of claim 12, further comprising the step of (i): verifying a CNV of the number of positive counts of target genes relative to the number of positive counts of normal genes obtained in the step (f) with the CNV result obtained in the step (h).

14. The method of claim 1, wherein the dsNA fragments in the samples are derived from single-stranded nucleic acid (ssNA).

15. The method of claim 14, wherein the ssNA is RNA-derived cDNA.

16. The method of claim 15, wherein steps (a) and (b) of the method are omitted when the ssNA is RNA-derived cDNA, and satisfied with the following condition that when forming the dsNA fragment from the ssNA which is derived from RNA, and both ends of the dsNA fragment are ligated with a double-stranded homogenous adapter, which is a complementary dsNA fragment having one oligonucleotide strand with 5′-phosphate and the other oligonucleotide strand with 3′-thymine (T) or 3′-uracil (U).

17. A kit for performing the method of claim 1, comprising:
(i) a double-stranded homogenous adapter as claim 1 defined;
(ii) primers, comprising a monotype bidirectional primer corresponding to the double-stranded homogeneous adapter, and a forward primer and a reverse primer both specific to a target gene;
(iii) probes, comprising probes corresponding to the forward primer and the reverse primer, and a plurality of probes;
(iv) enzymes, comprising uracil-specific excision reagent enzyme (USER enzyme);
(v) PCR reagents; and
(vi) detection reagents.


SEQUENCE LISTING (10 sequences)

SEQ ID NO: 1 - DNA, 21 bp, Artificial Sequence
  Primer
  misc_feature (1)..(21)
cgaaggcatcctggctagagg

SEQ ID NO: 2 - DNA, 25 bp, Artificial Sequence
  Probe
  misc_feature (1)..(25)
ccgccctaatccttttgcagccctt

SEQ ID NO: 3 - DNA, 24 bp, Artificial Sequence
  Primer
  misc_feature (1)..(24)
acagcaacaacagaaacgtgtagc

SEQ ID NO: 4 - DNA, 27 bp, Artificial Sequence
  Probe
  misc_feature (1)..(27)
catgtgcgcattcttcccctctctcct

SEQ ID NO: 5 - DNA, 16 bp, Artificial Sequence
  Haif adapter(HA)
  misc_feature (1)..(16)
gtcggagtctagcgct

SEQ ID NO: 6 - DNA, 17 bp, Artificial Sequence
  Half adapter(HA)
  misc_feature (1)..(17)
agcgctagactccgact

SEQ ID NO: 7 - DNA, 17 bp, Artificial Sequence
  Half adapter(HA)
  misc_feature (1)..(17)
agcgcuagacuccgacu

SEQ ID NO: 8 - DNA, 20 bp, Artificial Sequence
  Primer
  misc_feature (1)..(20)
gtgctctccaattctcgcct

SEQ ID NO: 9 - DNA, 19 bp, Artificial Sequence
  Primer
  misc_feature (1)..(19)
cccgtgatggcctagagga

SEQ ID NO: 10 - DNA, 29 bp, Artificial Sequence
  Probe
  misc_feature (1)..(29)
cactaaagttccttccaccctctcctggg